(19) United States
(12) Patent Application Publication
(10) Pub. No.: US 2008/0039770 A1
(43) Pub. Date: Feb. 14, 2008

(54) Devices with Photocatalytic Surfaces and Uses Thereof

(75) Inventors: Richard W.A. Francis, White Bear Lake, MN (US); Carl A. Schu, Plymouth, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 11/836,841

(22) Filed: Aug. 10, 2007

Related U.S. Application Data
(60) Provisional application No. 60/822,069, filed on Aug. 10, 2006.

(51) Int. Cl. A61M 37/00

(52) U.S. Cl. 604/20; 600/316; 604/501

(57) ABSTRACT

The present invention is directed to a device, such as a medical device, comprising at least one photocatalytic layer and at least one source of electromagnetic radiation, such as an LED or an electroluminescent material and methods of use. In some embodiments, the device includes an electrically conductive layer, such as an optically transparent conductive oxide, that is adapted to be biased from a voltage source. A method for increasing the energy efficiency of a photocatalytic surface comprises electrically biasing a transparent conductive oxide layer. A method for illuminating a complex three-dimensional surface comprises illuminating a photocatalytic layer with electromagnetic radiation from an electroluminescent layer. The devices and methods of the invention are useful, for example, for removing or preventing the formation of organic matter on a sensor window.

### PRIORITY

This application claims priority to provisional U.S. application Ser. No. 60/822,069, filed Aug. 10, 2006, entitled “Implantable Devices With Photocatalytic Surfaces” which is herein incorporated by reference in its entirety.

### FIELD OF THE INVENTION

This invention relates to devices having photocatalytic surfaces that are responsive to electromagnetic stimulation and uses thereof. Specifically, the invention relates to implantable devices having photocatalytic surfaces that form superhydrophilic surfaces in response to electromagnetic radiation.

### BACKGROUND OF THE INVENTION

The use of implants in humans and other mammals for medical purposes has become common. Problems associated with implantation of any foreign matter into humans or other mammals include infection and rejection by the immune system. Certain biomaterials used in implants may help to prevent rejection of the implant by the immune system and/or assist the body in fighting off organisms that cause infection. Attempts to limit an implant's likelihood of producing an infection or of being rejected by the immune system have been made with limited success.

### SUMMARY OF THE INVENTION

The present invention is directed to devices, such as implantable medical devices, having photocatalytic surfaces that are responsive to electromagnetic stimulation and uses thereof.

In one aspect, the invention is directed to a device, such as an implant, which includes a photocatalytic layer disposed on an electrically conductive layer, wherein the conductive layer is adapted to be electrically biased.

In one embodiment, the device includes an insulating layer located between the photocatalytic layer and the electrically conductive layer.

In another embodiment, the device includes an insulating layer, wherein the electrically conductive layer is located between the photocatalytic layer and the insulating layer.

In another embodiment, the device includes a voltage source adapted to electrically bias the electrically conductive layer.

In another embodiment, the photocatalytic layer includes a laminate which includes at least one metal and at least one catalytic agent.

In another embodiment, the photocatalytic layer includes a composite which includes at least one metal and at least one catalytic agent. The composite can include shelled particles or coated particles or mixtures thereof.

In another embodiment, the implant includes at least one light source adapted to provide electromagnetic radiation to a photocatalytic layer. In one embodiment, the at least one light source is adapted to provide electromagnetic radiation to the photocatalytic layer at a non-normal angle of incidence, such as from the side.

In another embodiment, the implant includes an electrically conductive layer that is at least partially transparent to electromagnetic radiation, specifically electromagnetic radiation provided to the photocatalytic layer from a light source.

In one embodiment, the light source is a light emitting diode (LED). In one embodiment the LED produces light selected from the group consisting of visible and ultraviolet.

In another embodiment, the electrically conductive layer includes SnO₂, In₂O₃, carbon nanotubes, conductive polymers, metal dispersions, conductive composite materials, colloidal silver, or mixtures thereof.

In another embodiment, the device includes a light sensitive diode adapted to receive a signal from outside the implant.

In another embodiment, the device includes a photovoltaic cell that may be adapted to convert light from a light source into electrical energy. The photovoltaic cell may also convert light that is unused by the photocatalytic layer into electrical energy, and this electrical energy may be used to recharge a battery or electrically bias an electrode.

In another embodiment, the device includes an induction coil connected to a rechargeable battery.

In another embodiment, the device includes a circuit board comprising a telemetry coil. In one embodiment, the circuit board is adapted to communicate with an external device and may regulate electrical energy supplied to a light emitting diode (LED). The circuit board may also communicate with an external device and regulate electrical energy supplied to an electrode.

In another embodiment, the device is at least partially enclosed by a housing comprising a hermetic seal, wherein the housing is electrically grounded by an in vivo environment contacting the housing.

In one embodiments, the device may be located inside a human or animal.

In another embodiment, the photocatalytic layer includes a material selected from the group consisting of TiO₂, NaTaO₃, ZnO, CdS, GaP, SiC, WO₃, ZnS, CdSe, SrTiO₃, CaTiO₃, KTaO₃, Ta₂O₅, ZrO₂, doped or non-doped, sensitized or non-sensitized, and mixtures thereof.

In another embodiment, the device is a sensor selected from the group consisting of an oxygen sensor, an electromagnetic radiation sensor, an impedance sensor, a pressure sensor, a protein sensor and a glucose sensor.

In another embodiment, the device is an oxygen sensor.

In another embodiment, the device includes a spectroscopy device.

In another embodiment, the device includes a sensor window.

In another embodiment, the device includes a transmitter adapted to transmit an outgoing sensor signal or a receiver adapted to detect an incoming sensor signal. In one embodiment, the transmitter is a light emitting diode and the receiver is an optical sensor.

In another embodiment, the device includes a reflective material, such as a mirror or parabolic reflector.

In another embodiment, the device includes a collimating lens, such as a focusing lens.

In another aspect, the present invention is directed to a method, which includes providing a medical implant which includes a photocatalytic layer and an electrically conductive layer, illuminating the photocatalytic layer with a light source and electrically biasing the electrically conductive layer.

In one embodiment, the implant of the method includes a sensor selected from the group consisting of an oxygen sensor, an electromagnetic radiation sensor, an impedance sensor, a pressure sensor, a protein sensor, and a glucose sensor.

In another embodiment, the implant of the method includes a spectroscopy device.

In another embodiment, the implant of the method includes a sensor window.

In another embodiment, the illumination and biasing steps are simultaneous.

In another embodiment, the biasing step occurs after the illumination step.

In another embodiment, the method includes illuminating the photocatalytic layer at a non-normal angle of incidence, such as from the side.

In another embodiment, organic matter is removed from a surface of the photocatalytic layer.

In another embodiment, the formation of an organic matter layer on a sensor window is prevented.

In another embodiment, the implant of the method includes any combination of the features described herein for the various device and implant embodiments.

In another aspect, the invention is directed to an implant which includes a photocatalytic layer and an electroluminescent layer

In one embodiment the implant includes an electrode layer disposed between the electroluminescent layer and the photocatalytic layer.

In another embodiment the implant includes an insulating layer disposed between the electroluminescent layer and the photocatalytic layer.

In another embodiment the electrode is optically transparent.

In another embodiment the electrode layer includes a conductive oxide, such as SnO₂, In₂O₃, carbon nanotubes, conductive polymers, metal dispersions, conductive composite materials, colloidal silver, or mixtures thereof.

In another embodiment the implant includes a distal electrode disposed between the electroluminescent layer and the photocatalytic layer, and a proximal electrode disposed between a base layer and the electroluminescent layer. In one embodiment, the proximal electrode and the distal electrode each include a transparent conducting oxide. In one embodiment the conductive oxides are the same, and in another embodiment they are different. In other embodiments, the distal electrode is transparent and the proximal electrode is not transparent.

In another embodiment, the electroluminescent layer is adapted to illuminate the photocatalytic layer.

In another embodiment, the electroluminescent layer includes quantum dots.

In another embodiment, the device is a sensor selected from the group consisting of an oxygen sensor, an electromagnetic radiation sensor, an impedance sensor, a pressure sensor, a protein sensor and a glucose sensor.

In another embodiment, the device is an oxygen sensor.

In another embodiment, the device includes a spectroscopy device.

In another embodiment, the device includes a sensor window.

In another aspect, the present invention is directed to a method including disposing an electroluminescent layer on a medical implant and illuminating a photocatalytic layer disposed on the medical implant with light from the electroluminescent layer.

In one embodiment, the implant of the method includes an electrode layer disposed between the photocatalytic layer and the electroluminescent layer.

In another aspect, the present invention is directed to a method for controlled delivery of a therapeutic agent, including providing a medical implant having one or more therapeutic agents disposed on a photocatalytic layer on the implant, and illuminating the photocatalytic layer with electromagnetic radiation, wherein the therapeutic agent comprises a drug, a protein, DNA, siRNA, or a virus that is modified to deliver a therapeutic gene, or mixtures thereof.

In one embodiment, the therapeutic agent is disposed within a matrix or discrete reservoir.

In another embodiment, the therapeutic agent is released by the photocatalytic reaction.

In another aspect, the invention is directed to a tissue scaffold adapted to grow cellular tissue which includes a photocatalytic layer having a surface upon which cellular tissue resides and whereby the surface is adapted to become more hydrophilic upon illumination with electromagnetic radiation.

In one embodiment, the the photocatalytic layer includes TiO₂.

In another embodiment, the tissue scaffold is adapted to release cellular tissue from the surface that becomes more hydrophilic upon illumination of the photocatalytic layer with electromagnetic radiation.

In another aspect, the invention is directed to a method including providing a tissue scaffold having a photocatalytic layer adapted to grow cellular tissue and illuminating the superhydrophilic layer.

In one embodiment, the superhydrophilicity of the photocatalytic layer is increased upon illumination.

In another embodiment the cellular tissue is more easily removed from the tissue scaffold upon illumination of the photocatalytic layer as compared to when the photocatalytic layer is not illuminated.

In another aspect, the invention is directed to a medical device including at least one superhydrophilic layer, and at least one waveguide layer, wherein the waveguide layer is adapted to distribute light from at least one light source to the at least one superhydrophilic layer.

In one embodiment, the medical device includes a light port disposed to receive a fiber optic cable from a light source.

In another embodiment, the medical device includes a catheter that may be a drainage catheter, therapy delivery catheter, or hydrocephalus shunt.

In another embodiment, the medical device includes a sensor including but not limited to an oxygen sensor, an electromagnetic radiation sensor, a glucose sensor, an impedance sensor, and a pressure sensor.

In another embodiment, the medical device includes a sensor window.

In another embodiment, the medical device includes a spectroscopy device.

In another aspect, the invention is directed to a method including providing a medical device comprising at least one photocatalytic layer and at least one waveguide layer, wherein the at least one waveguide layer is adapted to distribute light from at least one light source to at least one photocatalytic layer; and illuminating the at least one photocatalytic layer with light from the waveguide layer, to make the photocatalytic layer superhydrophilic.

In one embodiment, the medical device becomes more superhydrophilic upon illumination of a photocatalytic layer.

In another embodiment, the photocatalytic layer is illuminated prior to or during insertion of the medical device into a human or animal.

In another embodiment, the photocatalytic layer is not illuminated when a medical device is in a desired location.

In another embodiment, the photocatalytic layer is illuminated prior to or during extraction of a medical device from a human or animal.

In another embodiment, the method includes steering a medical device to a desired location by intermittently illuminating and not illuminating the photocatalytic layer.

In another embodiment, the medical device of the method is a catheter, an oxygen sensor, an electromagnetic radiation sensor, a spectroscopy device, an impedence sensor, a pressure sensor or a glucose sensor.

In another embodiment, the medical device includes a sensor window.

In another aspect, the invention is directed to a medical device including a photocatalytic layer, wherein the photocatalytic layer includes a composite or laminate, wherein the composite or laminate comprises at least one metal and at least one catalytic agent.

In one embodiment, the the catalytic agent includes at least one semiconductor.

In another embodiment, the catalytic agent includes at least one Perovskite compound.

In another embodiment, the metal includes platinum group metals, silver, gold, aluminum, iron, or mixtures thereof.

In another embodiment, the composite or laminate includes shelled particles or coated particles.

In another embodiment, the composite or laminate includes TiO₂—Au, ZnO—Pt, or TiO₂—CdSe.

In another aspect, the invention is directed to an implant or medical device including a base material having an outer surface, a wave guide, and a photocatalytic layer. The wave guide comprises an inner surface and an outer surface, wherein the inner surface of the wave guide may be disposed adjacent the outer surface of the base material. The photocatalytic layer comprises a semiconductor oxide having an inner surface disposed adjacent the outer surface of the wave guide.

In another aspect, the invention is directed to an implant or medical device including a base material having an outer surface, a waveguide and a light port. The wave guide includes an inner surface disposed adjacent the outer surface of the base material and the light may be port coupled to the waveguide and adapted to receiving a light signal.

In another aspect, the invention is directed to an implant or medical device including a photocatalytic layer having a semiconductor oxide that may be doped. Furthermore, the photocatalytic layer may have an inner surface and an outer surface, and the outer surface of the semiconductor oxide may be doped. Suitable dopants may include without limitation, ion-implanted metals, vanadium, chromium, nitrogen, Nd⁺³, Pd⁺², Pt⁺⁴, and Fe⁺³. Moreover, a photocatalytic surface may comprise titania, wherein titania is a bulk layer.

In another aspect, the invention is directed to an implant or medical device including a semiconductor oxide having an outer surface that has a light absorption maximum at a wavelength of at least 400 nm. According to some embodiments, a semiconductor oxide comprises a composite layer including a waveguide. The semiconductor oxide may further comprise a reflective layer disposed upon the composite layer.

In another aspect, the invention is directed to an implant or medical device including a composite material comprising a first material and a second material. The first material has a transmissivity of at least 50% when exposed to a predetermined wavelength of light; and the second material has photocatalytic activity when exposed to the predetermined wavelength of light. The first material may comprise silica or alumina or mixtures thereof The second material my comprise titania.

In another aspect, the invention is directed to an implant, such as a biomedical implant including a photocatalytic surface and a light source adapted to irradiate the photocatalytic surface. The light source and the photocatalytic surface are configured such that the irradiation of the photocatalytic surface with the light source produces a photocatalytic effect.

In another aspect, the invention is directed to a photocatalytic system including an implant having a photocatalytic surface and an external light source adapted to irradiate the photocatalytic surface of the implant.

In another aspect, the invention is directed to a method of performing a procedure upon a patient, comprising the acts of providing a cylinder comprising an outer surface having a photocatalytic layer, advancing the cylinder through a tissue of the patient, and, irradiating the photocatalytic layer of the cylinder so that at least a portion of the irradiated photocatalytic layer may be in contact with the tissue. In one embodiment of the invention, the cylinder may be advanced through a dermal layer causing microbes such as Staph epidermis to attach to the photocatalytic layer. Upon irradiation of the photocatalytic layer, at least a portion of the microbes may be killed. In addition, the cylinder may comprise a cannula having proximal and distal ends or a dilator having a closed distal end.

In another aspect, the invention is directed to a cylinder or catheter having an inner barrel and a light source disposed within the inner barrel and may further include a base material made of a UV transmissive material. The cylinder may also comprise a fluid transmission channel that enters the cylinder at the proximal end portion of the cylinder and exits along the intermediate portion of the cylinder at the outer surface.

In another aspect, the invention is directed to a cylinder for penetrating a tissue of a patient, including a distal end portion adapted to penetrate tissue, an elongated intermediate portion, a proximal portion, a base material forming an outer surface; and a photocatalytic layer disposed upon at least a portion of the outer surface.

In another aspect, the invention is directed to a sterilization system including a cylinder for penetrating a tissue of a patient and a light transmission device coupled to the proximal end portion of the cylinder. The cylinder comprises a distal end portion adapted to penetrate tissue, an elongated intermediate portion, a proximal portion, a base material forming an outer surface, and a photocatalytic layer disposed upon at least a portion of the outer surface of the base material.

In another aspect, the invention is directed to a shunt device including a structural component housed within a tubing. In one embodiment, the tubing comprises an outer tube having an outer wall and an inner wall, a photocatalytic layer attached to the inner wall of the outer tube, and a light port. The outer tube may comprise silicone. In another embodiment, the structural component includes a baseplate having a first surface, and a photocatalytic layer disposed upon a first portion of the first surface of the baseplate. The structural component may comprise a valve component disposed upon a second portion of the first surface of the baseplate.

In another aspect, the invention is directed to a method of performing a procedure upon a patient including the acts of providing a shunt comprising a structural component housed within a tubing having an inner surface, wherein at least one of the structural component and the inner surface of the tubing has a photocatalytic layer disposed thereon, implanting the shunt in the patient, and irradiating the photocatalytic layer.

In another aspect, the invention is directed to a wave guide including a material selected from the group consisting of alumina, silica, CaF, titania, single crystal-sapphire, polyurethane, epoxy, polycarbonate, nitrocellulose, polystyrene and PCHMA.

Other features, objects and advantages of the present invention will be in part apparent to those skilled in art and in part pointed out hereinafter. All references cited in the instant specification are incorporated by reference for all purposes. Moreover, as the patent and non-patent literature relating to the subject matter disclosed and/or claimed herein is substantial, many relevant references are available to a skilled artisan that will provide further instruction with respect to such subject matter.

### BRIEF DESCRIPTION OF THE DRAWINGS

The accompanying drawings are not intended to be drawn to scale. In the drawings, each identical or nearly identical component that may be illustrated in various figures may be represented by a like numeral. For purpose of clarity, not every component may be labeled in every drawing. In the drawings:

FIG. 1 is a cross-section of a surface portion of a medical implant with a photocatalytic layer according to an embodiment of the present invention.

FIG. 2 is a cross-section of a surface portion of a medical implant having a photocatalytic layer and a dopant according to an embodiment of the present invention.

FIG. 3 is a cross-section of a portion of an implant having an intermediate waveguide layer and an upper photocatalytic layer according to an embodiment of the present invention.

FIG. 4 is a cross-section of a portion of an implant having a waveguide layer, a photocatalytic layer, and a reflective layer according to an embodiment of the present invention.

FIG. 5 is an implant having a lower waveguide layer, an intermediate partially reflective layer, and an outer doped photocatalytic layer according to an embodiment of the present invention.

FIG. 6 is a cross-section of an implant having a light port and a light source that may be external to the body according to an embodiment of the present invention.

FIG. 7 is a cross-section of an implant that may be powered by an ex vivo RF link and has an internal light source according to an embodiment of the present invention.

FIG. 8 illustrates a device with internal light source and electrically-biased transparent conductive layer according to an embodiment of the present invention.

FIGS. 9A, 9B, 9C, and 9D illustrate side illumination according to an embodiment of the present invention.

FIG. 10 illustrates an implant comprising a photocatalytic layer and photovoltaic cells.

FIG. 11 illustrates an implant device in an in vivo environment having a photocatalytic layer and an electrode layer.

FIG. 12 illustrates a finite element of a photocatalytic device with an electroluminescent layer according to an embodiment of the present invention.

FIG. 13 is a cross-section of a tissue scaffold according to an embodiment of the present invention.

FIG. 14 is a cross-section of a catheter according to an embodiment of the present invention.

FIG. 15 depicts a schematic of reaction mechanisms leading to pronounced photocatalysis and superhydrophilicity.

FIG. 16 depicts a schematic showing fluorescently labeled BSA at the surface of TiO2 coated silica specimen irradiated with UV from below for demonstrating photocatalytic effect.

FIG. 17(a) depicts fluorescently labeled BSA adhered to a control surface of TiO₂ coated silica with no UV illumination.

FIG. 17(b) depicts fluorescently labeled BSA at the surface of UV irradiated TiO2 coated silica specimen.

### DETAILED DESCRIPTION OF THE INVENTION

The following description is intended to convey a thorough understanding of exemplary embodiments of the invention by providing a number of specific embodiments and details involving photocatalytic implantable device surfaces responsive to electromagnetic stimulation. It is understood, however, that the present invention is not limited to these specific embodiments and details, which are exemplary only. It is further understood that one possessing ordinary skill in the art, in light of known systems and methods, would appreciate the use of the invention for its intended purposes and benefits in any number of alternative embodiments.

The phraseology and terminology used herein is for the purpose of description and should not be regarded as limiting. The use herein of “including,” “comprising,” “having,” “containing,” “involving,” and the like is meant to encompass the items listed thereafter and equivalents thereof as well as additional items.

The terms “light” and “illumination” as used herein means any form of electromagnetic radiation, including without limitation, ultraviolet radiation (UV), visible light, and infrared radiation (IR).

The term “illuminate” or “irradiate” as used herein means to cause electromagnetic radiation to contact or pass through all or a part of the illuminated subject.

The terms “transparent” or “optically transparent” as used herein mean permeable or semi-permeable to electromagnetic radiation.

“Medical device” as used herein means any instrument, apparatus, implement, machine, contrivance, implant, or other similar or related article, including a component part, or accessory which is intended for use in the diagnosis of disease or other conditions, or in the cure, mitigation, treatment, or prevention of disease, in man or other animals, or is intended to affect the structure or any function of the body of man or other animals.

An “implantable medical device,” “implant,” “medical implant,” or “implant device” as used herein means any medical device that resides either fully or partially within the body either temporarily or long-term when performing its intended function. An “implantable medical device,” “implant,” “medical implant,” or “implant device” may comprise but is not limited to shunts for the treatment of hydrocephalus and other conditions, drainage, delivery and ablation catheters, leads, stylets, introducers, cardiovascular stents, abdominal aortic stents and stent-grafts, non-cardiovascular stents including nasal and esophageal, vascular and non-vascular grafts, stent-grafts and fistulas, surgical mesh, patches, and sutures, surgical instruments, cardiac pacemakers, implantable cardioverter defibrillators (ICDs), implantable heart monitors, cardiac ablation catheters and mapping devices, biological pacemakers, and associated leads, sensing and pacing electrodes, cardiac surgery devices including blood oxygenators, blood pumps, beating heart surgical tools and cannula for performing heart bypass procedures, bioprosthetic or mechanical heart valves either replaced by surgical means or delivered percutaneously, internal or external pumps, syringes, catheters, needles, cannula or other infusion means for delivering therapeutic agents including cells, genes, polynucleotides, proteins, small molecules, or other therapeutic agents to the cardiac, neural, spinal, cerebrospinal, vascular, or lymphatic systems, or to other organs or tissues, transdermal, nasal, sinus, or inhalation devices for delivery of therapeutic agents to subdermal, sinus, brain or lung tissue, intraspinal infusion devices for the treatment of spasticity, multiple sclerosis, brain injury, spinal cord injury and stroke or other conditions, hepartic arterial infusion devices for the treatment of cancer or other conditions, external or internal monitors or sensors to monitor physiological parameters including blood pressure, blood oxygenation, other blood gases, analytes including glucose and potassium and sodium ion concentration, and other physiological parameters whether alone or in combination with other medical devices such as drug pumps or pacemakers, devices for performing image-guided cardiac, cranial, spinal, ENT or other medical procedures, including catheters to be inserted into the body, devices for treatment of Benign Prostatic Hyperplasia (BPH), devices for the diagnosis and/or treatment of Gastroesophageal Reflux Disease (GERD), including pH and mobility testing devices and implantable gastric electrical stimulators for the treatment of gastroparesis, devices for urodynamic testing and for treating voiding dysfunction, or bladder control problems, sacral nerve stimulators and other neurological stimulation devices for the treatment of pain, dystonia, and other conditions, stimulation devices for the treatment of obesity, sleep apnea and other conditions, neurological leads for sensing or delivery electrical therapy in the brain, musculoskeletal and other systems, and devices for the treatment of orthopaedic conditions including spinal fusion devices, disc replacement devices, and fracture fixation devices.

“Photocatalytic layer” as used herein means a layer comprising a photocatalytic material whereby illumination of the photocatalytic material with electromagnetic radiation of an appropriate wavelength causes the photocatalytic material to act as a catalyst or to increase its catalytic activity. When the photocatalytic material is illuminated in the presence of water and oxygen in a biological milieu, the catalytic activity comprises generation of reactive oxygen species (ROS) that may include but are not limited to hydroxyl and perhydroxyl radicals and superoxide anion. Generation of ROS at the photocatalytic layer may result in an increase in hydroxylation of the photocatalytic surface, thereby rendering the surface more hydrophilic. When the photocatalytic surface is sufficiently hydroxylated such that a water contact angle measurement approaches zero the surface is said to exhibit superhydrophilicity and may inhibit the binding or retention of organic matter including proteins, cells and tissue. Another consequence of generating ROS at the photocatalytic layer may be to cause reaction between the ROS and resident or proximal organic matter, tissue or cells, including bacteria, leading to removal of adherent biological matter at the photocatalytic layer and/or destruction of bacteria or occlusive cells or tissue in the vicinity of the photocatalytic layer.

“Electrically bias” as used herein, means applying a voltage to one or more elements of the device to establish a desired operating point. For example, the electrically conductive layer or photocatalytic layer can be electrically biased in order to promote or retard charge separation or to promote or retard electron hole recombination and annihilation in the photocatalyst. The electrically conductive layer or photocatalytic layer can also be electrically biased in order to drive a light source for subsequent illumination of the photocatalytic layer, or to promote functioning of the integral electrical/electronic components.

A photocatalytic layer comprising one or more photocatalytic materials can be dye-sensitized such that the photocatalytic layer exhibits photocatalytic activity at longer wavelengths of illuminated light than without dye-sensitization using dyes whose absorbance occurs at longer wavelengths than the base photocatalytic materials. Suitable dye-sensitizers include erythrosine, rose bengal, and metal phthalocyanines including copper phthalocyanine. The dye can be adsorbed to the photocatalytic material or admixed with the photocatalytic material within the photocatalytic layer.

Titanium dioxide (TiO₂) in appropriate forms such as thin films of anatase may be made to exhibit pronounced photocatalytic and superhydrophilic behavior when irradiated with specific wavelengths of electromagnetic radiation. This effect offers the basis for biological-shedding surfaces for a variety of implantable medical device applications.

According to some embodiments, a photocatalytic layer comprising a semiconductor material (e.g., a metal oxide such as TiO₂) may be used for photocatalytic purposes to assist in the prevention and elimination of infection on an implant device. Titanium dioxide has been shown to have photocatalytic activity for generating reactive oxygen species that are lethal to pathogens. In various embodiments the photocatalytic layer comprises titania in the anatase form.

Illumination of TiO₂ with electromagnetic radiation of the appropriate wavelength causes promotion of electrons from the valence band to the conduction band. This effect may be greater in the anatase form of TiO₂ than in the more stable rutile form. Upon promotion to the conduction band, the electrons leave behind positively charged holes in the crystal lattice. While some of these holes are immediately annihilated by recombination with electrons, a portion manage to migrate to the surface of the TiO₂ where they are available to react with oxygen and water to form reactive oxygen species including hydroxyl and perhydroxyl radicals. These powerful bioactive radicals are capable of destroying cell membranes and denaturing proteins. When employed in some embodiments such as medical implants, these reactive oxygen species may act to destroy pathogens including bacteria, viruses, and molds close to the surface of the implant, thereby reducing or preventing infection, or reducing or preventing the formation of organic matter that would otherwise obscure the surface.

A concurrent superhydrophilic effect occurs in vivo as a consequence of wide scale hydroxylation at the surface. Subsequent hydrogen bonding promotes a thin continuous thin layer of water causing the contact angle to diminish towards zero.

These effects may be demonstrated by introducing aliquots of fluorescently labeled bovine serum albumin (BSA) directly onto a TiO₂ surface and irradiating the surface with UV light at a wavelength of 365 nm from below. Irradiation of TiO₂ at this wavelength promotes a photocatalytic reaction leading to a surface contact angle approaching zero and generation of reactive oxygen species that degrade or dissuade proteins adsorbing at the surface.

It has further been discovered that when the illuminated photocatalytic layer is disposed on an electrically biased transparent conductive oxide layer, the electrons in the conduction band are drawn toward the electrically biased surface, allowing a greater number of holes to migrate to the surface of the photocatalytic layer to react to create reactive oxygen species. Therefore, by retarding electron-hole recombination in this manner, it may be possible to increase the efficiency of the photocatalytic reaction.

In some embodiments an electroluminescent material may be used as a light source for photocatalysis. The use of such electroluminescent materials facilitates the transfer of light to complex 3-dimensional surfaces. Indeed, electroluminescent material may be deposited through spraying, dip coating, spin coating, printing (transfer, screen, inkjet, laser assisted), vapor deposition, physical deposition, and physical adherence including gluing onto a wide variety of complex surfaces.

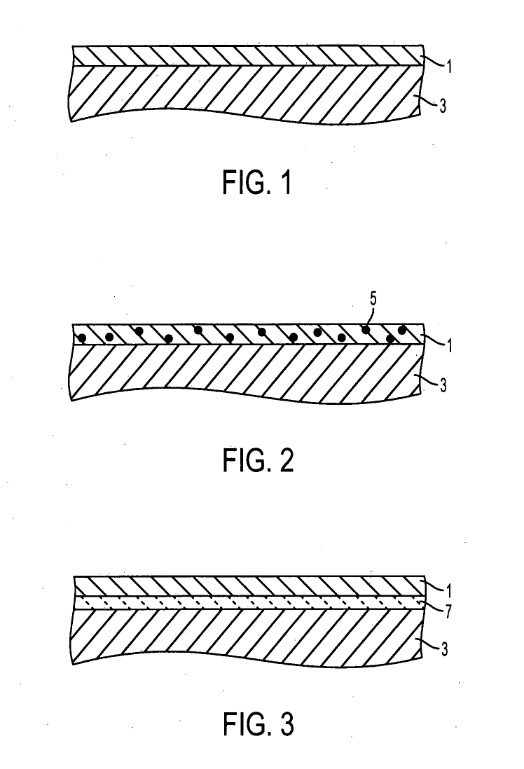

Referring now to FIG. 1, there is shown an embodiment having a photocatalytic layer 1 disposed upon a base layer 3. The photocatalytic layer 1 may comprise a semiconductor oxide or mixture of semiconductor oxides that without limitation may comprise TiO₂, NaTaO₃, ZnO, CdS, GaP, SiC, WO₃, ZnS, CdSe, SrTiO₃, CaTiO₃, KTaO₃, Ta₂O₅, ZrO₂, doped or non-doped, sensitized or non-sensitized, or mixtures thereof. Base layer 3 provides structural support for photocatalytic layer 1 and may comprise any suitable material for such purpose, as is readily apparent to one of skill in the art.

The photocatalytic layer 1 may be deposited on the base layer 3 using chemical vapor deposition techniques such as atomic layer disposition (ALD), atomic layer epitaxy (ALE), assisted CVD, and metalorganic vapor phase epitaxy; physical vapor deposition techniques such as high velocity oxygen fuel, pulsed laser deposition, sputtering, arc-PVD, EBPVD, plasma spraying, electroplating, and low-pressure plasma spraying (LPPS); other techniques such as evaporation, anodizing, ion beam assisted deposition (IBAD), magnetron sputtering, molecular beam epitaxy, slurry or dye techniques, sintering technique, sol-gel, and sputter ion plating; and other techniques known to those of skill in the art or combinations thereof. The ALD method may be used to deposit photocatalytic layer 1 to various thicknesses, including thin layers on the nano-layer scale, and the crystal phase of the TiO₂ may be controlled through temperature manipulation.

Semi-conductor photocatalytic reactions rely on illumination of a semiconductor with electromagnetic radiation of energy greater than the band gap of the material being illuminated. The band gap is the energy gap separating the semiconductor's conduction band from its valence band. The energy to do this work can be calculated by

$$\begin{matrix}λ = \frac{h c}{E} . & Equation 1 \\ E α \frac{1}{λ} . & Equation 2\end{matrix}$$

Wherein: λ=wavelength

h=Plank's constant

c=speed of light in a vacuum

E=energy

It will be appreciated by those of skill in the art that these equations may be used to determine the wavelength of electromagnetic radiation necessary to promote photocatalysis using a given semiconductor or to determine semiconductors suitable for use as photocatalysts with given wavelengths of electromagnetic radiation.

Referring now to FIG. 2, there is shown an embodiment having a base layer 3 and a photocatalytic layer 1, wherein the photocatalytic layer additionally comprises a dopant 5. Doping of the photocatalytic layer may be achieved by sputtering or any other suitable method known to those of skill in the art. Doping allows the use of visible light to produce a photocatalytic effect through tuning of the band gap. According to the present invention, dopants may include, but are not limited to, nitrogen, sulfur, carbon, fluorine, vanadium, neodymium, and silver, or mixtures thereof.

Referring to FIG. 3, there is shown an embodiment having a photocatalytic layer 1, a base layer, 3, and a waveguide 7. The waveguide 7 may comprise a partially light reflective or transmissive material and may be adapted to distribute light from a light source to the photocatalytic layer 1. The use of a waveguide 7 may further allow light to be evenly and efficiently distributed to the photocatalytic layer 1 from inside the device. The waveguide 7 may comprise a continuous or local layer at the surface of the device or at the surface of any integral or ancillary components employed in the device. Moreover, waveguide 7 may comprise a discrete component attached or made fast to the device and/or ancillary components therein. In these multiple forms, of which, limited examples are described above, it can be appreciated that there are many ways to incorporate a waveguide into the device system, the method chosen will depend upon the nature of the waveguide and the material chosen for its fabrication. For coatings, this may comprise: chemical vapour deposition techniques such as atomic layer disposition (ALD), atomic layer epitaxy (ALE), assisted CVD, and metalorganic vapour phase epitaxy; physical vapour deposition techniques such as high velocity oxygen fuel, pulsed laser deposition, sputtering, arc-PVD, EBPVD, plasma spraying, electroplating, and low-pressure plasma spraying (LPPS); other techniques such as evaporation, ion beam assisted deposition (IBAD), magnetron sputtering, molecular beam epitaxy, slurry techniques, sintering techniques, sol-gel, and sputter ion plating, spraying, dipping, coating, spinning, casting, molding, overlaying and/or any combination of these methods and other techniques known to those of skill in the art. For the purpose of attaching and/or incorporating a waveguide component or subassembly into the device system, any suitable form of attachment or affixation may be used, including: any form of jointing, screw or bayonet fittings, any form of mechanical fixation including the use of fasteners; any form of molding or overmolding or insert molding, welding using thermal or ultrasonic energy by means such as electron beam, ultrasound, and laser; any form of cohesion or adhesion, including adhesive agents such as glue.

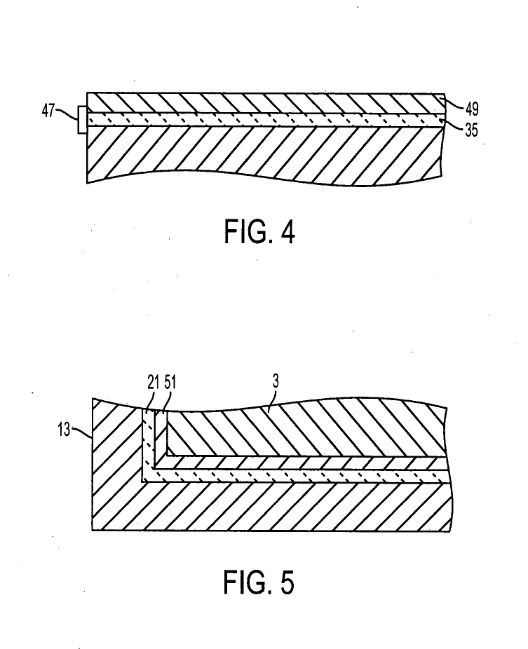

Referring to FIG. 4, there is shown an embodiment wherein a reflective surface 47 may be positioned at an end opposite where light enters a waveguide 35. Reflective surface 47 may be adapted to reflect light back into waveguide 35 and ultimately into the photocatalytic layer 49. For example, electromagnetic radiation exiting waveguide 35 may partially or completely pass out of waveguide 35 without contacting photocatalytic layer 49, and the use of a reflective surface may be provided to reflect that electromagnetic radiation into the photocatalytic layer. Such an embodiment provides the advantage of increased energy efficiency because it directs the maximum amount of light onto the photocatalytic surface.

Referring to FIG. 5, there is shown a multi-layered device which may comprise a base material 3 supporting a waveguide layer 21, a reflective layer 51, and a photocatalytic layer 13. The reflective layer may comprise a metallized mirrored surface and may reflect light from waveguide layer 21 to more effectively distribute light into photocatalytic layer 13.

It will be appreciated that other light-related components known to those of skill in the art that are designed to manipulate light and allow light to reach remote surfaces of a device may also be used to deliver light to the waveguide and are also contemplated by the various embodiments of the present invention.

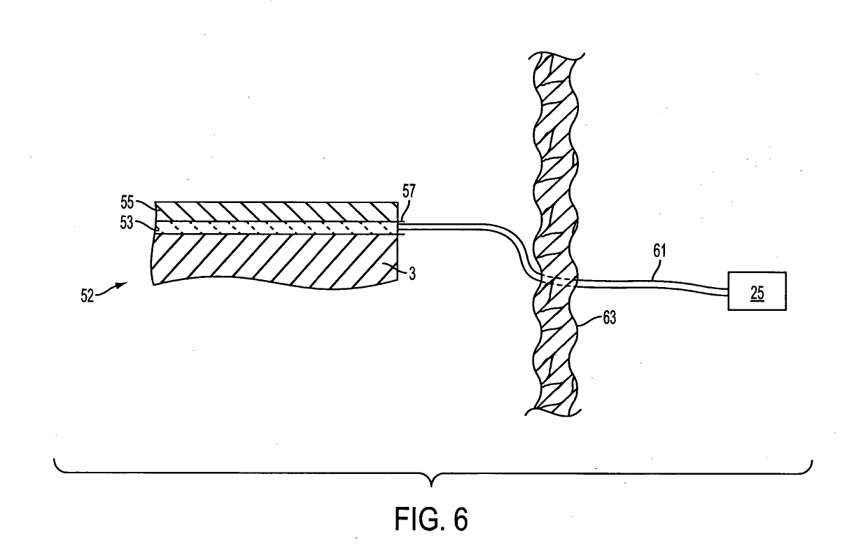

Referring to FIG. 6, there is shown a medical implant 52, which may comprise base material 3 supporting a waveguide 53 and a photocatalytic surface 55. The implant may also comprise a light port 57 adapted to receive the distal end 59 of fiber optic cable 61. The fiber optic cable 61 transports light from the light source 25 to the waveguide 53 by passing through skin, an orifice, an opening, a fistula, or any other access point to the body whether artificial or natural. The photocatalytic layer 55 receives the light from the waveguide 53 and may facilitate sterilization and disinfection of the surface of the implant device or may improve the ease of insertion or removal of the device through or from any natural or artificial opening into which the device may be inserted or embedded.

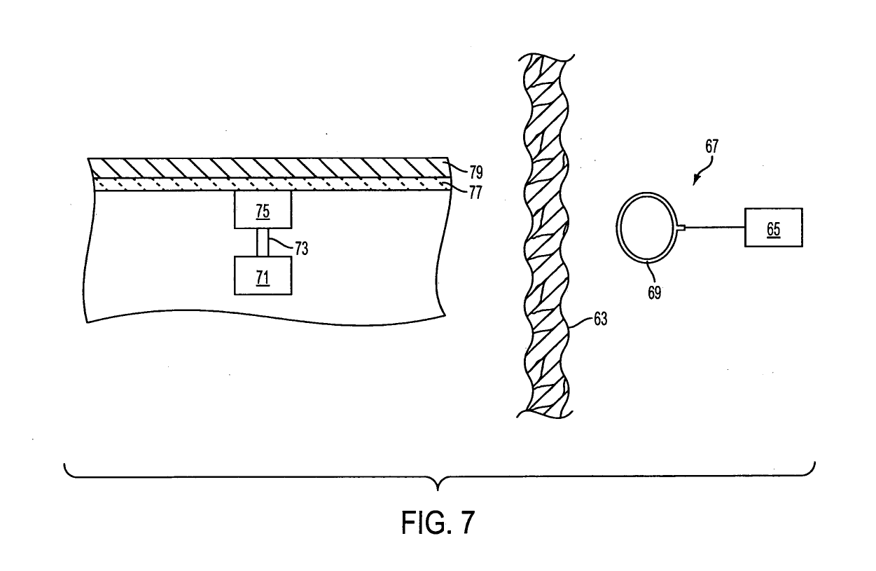

Referring to FIG. 7, there is shown a medical implant 62, which may comprise an internal light source. External control 67 may comprise an RF energy source 65 that provides power to an external antenna 69. External antenna 69 may be electromagnetically coupled to internal antenna 71, which may comprise an induction coil (not shown). Electricity travels from internal antenna 71 through conductor 73 to illuminate the light emitting diode (LED) 75. Light from LED 75 may be transferred to the waveguide layer 77, which disperses the light to the photocatalytic layer 79, thereby sterilizing and disinfecting the medical implant.

In some embodiments of the invention, the medical device may comprise an internal power source such as a battery (not shown), which may be controlled by an internal receiver capable of receiving control signals from outside the body.

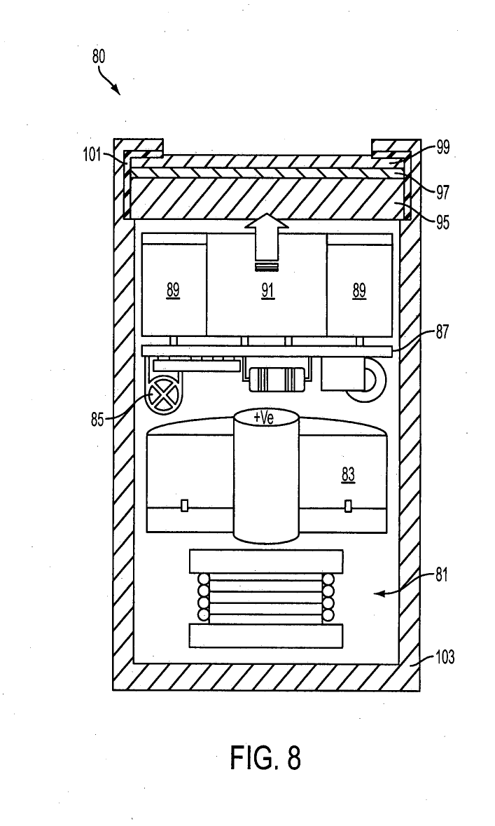

Referring to FIG. 8 there is shown a cross-section of a device 80 comprising a hermetically sealed device housing 103 with dielectric insulator 101 and an induction coil 81 capable of remote charging rechargeable battery 83. Furthermore, the implant device may comprise a circuit board 87 including an RF receiver and at least one transmission and receiver telemetry coil 85 adapted to communicate with an external controller (not shown) via telemetry. Electrical energy stored in rechargeable battery 83 may be regulated by circuit board 87 and may also be available to power light source 91 upon communication between circuit board 87 and an external controller via telemetry coil 85. Light sensitive diode 89 may be adapted to receive electromagnetic radiation signals if the device 80 is employed as a sensor. Without limitation, light source 91 may comprise one or more light emitting diodes (LEDs).

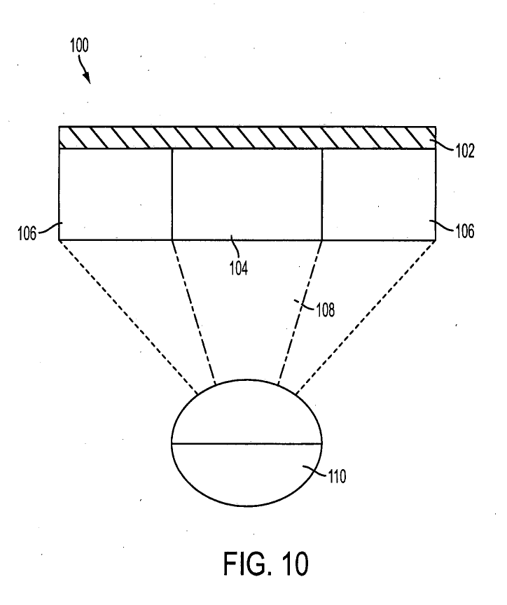

The device 80 may also comprise a support layer 95 which may comprise transparent sapphire crystal (Al₂O₃), borosilicates, aluminosilicates, SiO₂, fused silica, quartz, or other compounds known to those of skill in the art. The support layer 95 may be chosen according the desired electromagnetic radiation transmission properties of the substance as known to those of skill in the art. Support layer 95 may provide support to an electrode or electrically conductive layer 97, which in some embodiments is transparent. A photocatalytic layer 99 may contact electrode 97, and may comprise a semiconductor oxide or mixture of semiconductor oxides that without limitation may comprise TiO₂, NaTaO₃, ZnO, CdS, GaP, SiC, WO₃, ZnS, CdSe, SrTiO₃, CaTiO₃, KTaO₃, Ta₂O₅, ZrO₂, doped or non-doped, sensitized or non-sensitized, or mixtures thereof. Electrode 97 may comprise transparent conductive oxides such as indium or tin oxides or doped combinations thereof such as SnO₂, In₂O₃, carbon nanotube films, conductive polymers, metal dispersions, conductive composite materials, colloidal silver or mixtures thereof. Electrode 97 may further comprise thin layers of conductive media or fine conductive meshes that do not obscure the net flux of outward illumination nor hinder the detection of an incoming signal. It will be appreciated by those of skill in the art that electrode 97 may be chosen to ensure high transparency to the desired wavelengths of electromagnetic radiation and may have high electrical conductivity. Photocatalytic layer 99, electrode 97, and support layer 95 need not be located in housing 103 as illustrated in FIG. 10, but may be located remotely in one or more devices and may be connected to light source 91 by a fiber optic cable or waveguide.

Electrode 97 promotes charge separation by attracting electrons toward its positively charged upper surface, thereby electrically biasing photocatalytic layer 99 and retarding electron-hole recombination. Device 80 may be grounded using the in vivo environment surrounding housing 103. Electrode 97 and photocatalytic layer 99 may be deposited on support layer 95 by electroplating, printing, spraying, chemical vapor deposition (CVD), physical vapor deposition (PVD), RF magnetron sputtering, condensation, ALD, from slurry suspensions or dyes and by other means known to those of skill in the art.

Light from light source 91 may pass through support layer 95 and electrode 97 to promote photocatalysis in photocatalytic layer 99. Electrode 97 may be connected to circuit board 87 and may receive power from rechargeable battery 83. If device 80 is to be employed as a sensor, it is contemplated that device 80 may further comprise a torus-shaped light sensitive diode 89 that may be used to detect incoming signals.

It is contemplated that the device 80 may be employed in a variety of partially or fully implanted, long term or temporarily-placed medical devices and may comprise, optical sensors, oxygen sensors (including oxygen sensors incorporated into ICD and IPGs), glucose sensors, impedance sensors, pressure sensors, protein sensors, Fabrey-Perot interferometers/etalons/resonators infrared spectrophotometers, ultrasonic detectors, shunts, and spectroscopic devices known to those of skill in the art. Indeed, the use of at least partially optically transparent layers such as support layer 95, electrode 97, and photocatalytic layer 99, is advantageous in providing antifouling windows for a variety of devices. It is further contemplated that device 80 may comprise more than one light source and may comprise one or more LEDs capable of producing electromagnetic radiation of appropriate wavelengths.

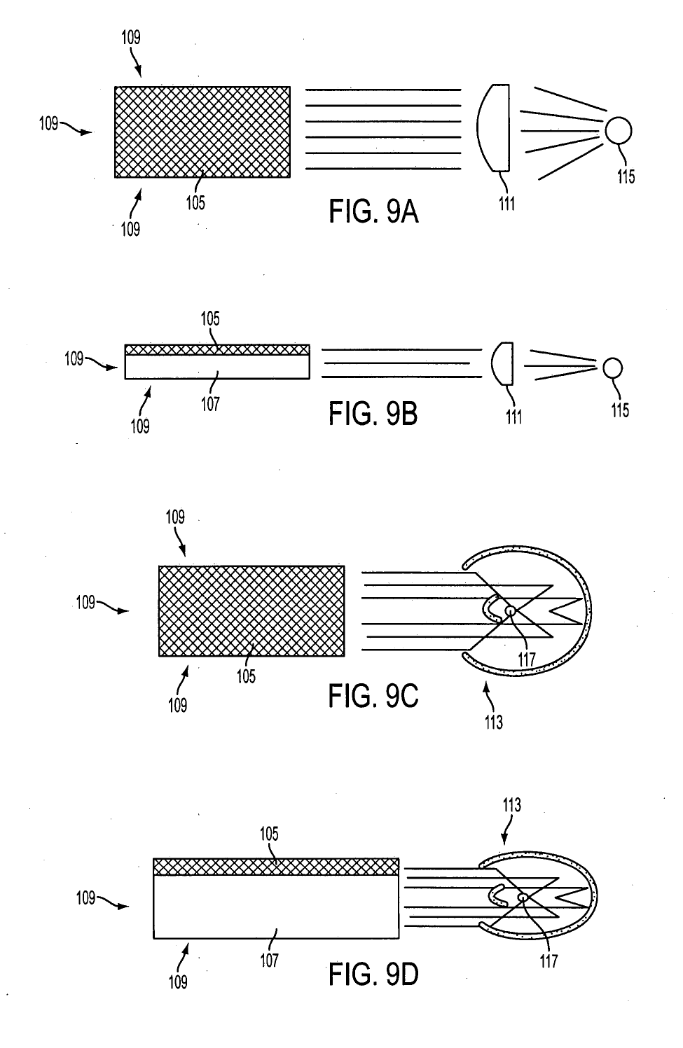

Referring to FIGS. 9A-D, there are shown embodiments wherein a photocatalytic layer 105 may be illuminated from a non-normal angle of incidence, such as from the side. FIGS. 9A and 9B are illustrations of the top and side views of the same device respectively. FIGS. 9C and 9D are illustrations of the top and side views of the same device respectively.

The photocatalytic layer 105 may be supported by transparent waveguide layer 107 having reflective material 109 disposed to reflect light (such as that which might otherwise exit or leak from the waveguide 107) back into waveguide 107 and eventually into photocatalytic layer 105, thereby increasing efficiency. With regard to FIGS. 9A and 9B, light from light source 115 passes through collimating lens 111 and illuminates the side of photocatalytic layer 105 and waveguide 107. With regard to FIGS. 9C and 9D, light from light source 117 may be directed by parabolic reflector 113 to illuminate photocatalytic layer 105 and waveguide 107.

In some embodiments, side illumination of the photocatalytic layer 105 results in very little light escaping from the photocatalytic surface. Such embodiments may be employed in in vivo environments where a low level of illumination or increased energy efficiency may be desired.

Furthermore, the edges (sides) of the photocatalytic layer 105 and the edges and bottom of waveguide 107 may be coated with a reflective material 109 and may be substantially perpendicular to the surface or may be parabolic in shape such that the incident light from the side is made to reflect, resulting in very little loss of light energy to the surrounding environment and a correspondingly high efficiency in reactive oxygen species production. This reduces the power consumption of the device.

As is shown in FIGS. 9A and 9B, side illumination may also be achieved by positioning the light source(s) to one side of the photocatalytic surface and then passing the light through a collimating lens, resulting in a light path that may be close to parallel with the surface. As is shown in FIGS. 9C and 9D, the light source may also be positioned at the focal point of a reflecting parabola, reducing wasted light energy, and decreasing power consumption.

Referring to FIG. 10, there is shown a schematic a photocatalytic device 100 comprising a photovoltaic cell 106. Photocatalytic layer 102 is disposed on transparent substrate 104. Light 108 from light source 110 may impinge upon transparent substrate 104 and photovoltaic cell 106 to promote photocatalysis in photocatalytic layer 102. It is contemplated that photovoltaic cell 106 may comprise a photodiode, photo-transducer, or other device for converting electromagnetic radiation into electrical energy known to those of skill in the art. Photovoltaic cell 106 may be torus-shaped and convert electromagnetic radiation not employed in photocatalysis into electrical energy. The electrical energy from photovoltaic cell 106 may be used to recharge a battery (not shown) connected to light source 110, or may be used to electrically bias an electrode (not shown). Conversion of light not used in photocatalysis into electrical energy may be used to improve the energy efficiency of the device.

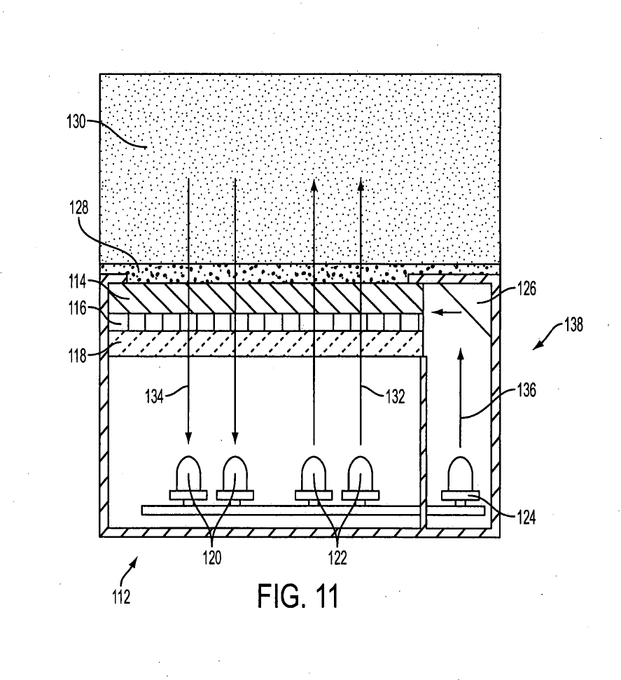

Referring to FIG. 11, there is shown a sensor device 112 adapted to remove or prevent the formation of an organic matter layer on transparent photocatalytic layer 114. Device enclosure 138 provides structural support for sensor device 112. Transparent substrate 118 supports transparent conductive layer 116 (which may be electrically biased as discussed with regard to other embodiments), and transparent photocatalytic layer 114, which collectively comprise the sensor window. Light 136 from light emitting diode (LED) 124 may be reflected by mirror 126 to illuminate transparent photocatalytic layer 114 from the side. LED may also be disposed such that it illuminates photocatalytic layer 114 directly without the use of mirror 126 (not shown). A photocatalytic reaction may then lead to the degradation and removal or prevention of the formation of organic matter layer 128 in in vivo environment 130. Sensor device 112 may further comprise one or more light emitting diodes (LEDs) 122 adapted to transmit an outgoing sensor signal 132 and one or more optical sensors 120 to detect incoming sensor signal 134. The removal or prevention of the formation of organic matter layer 128 may facilitate the transmission of outgoing sensor signal 132 and the receipt of incoming sensor signal 134. Sensor device 112, may be employed to detect a variety of in vivo conditions including blood oxygenation and glucose concentration.

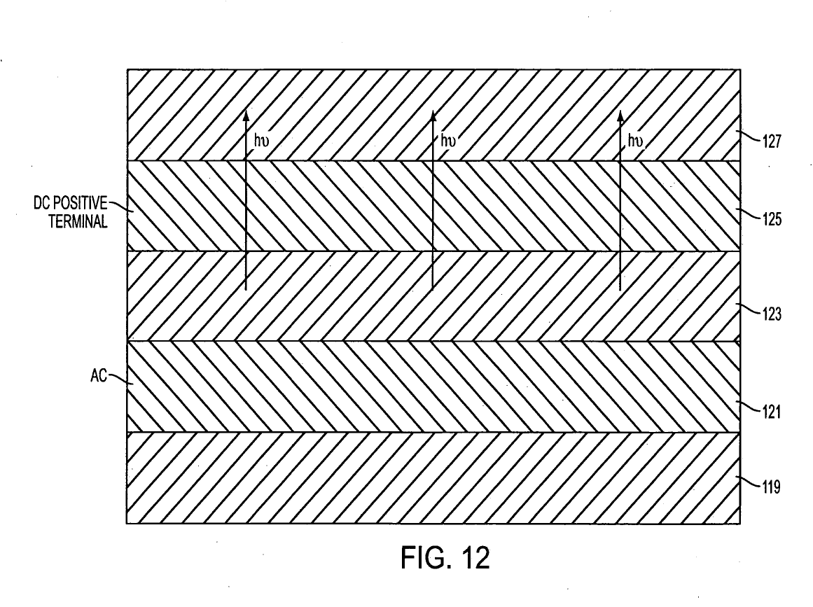

Referring to FIG. 12, there is shown a finite element of a photocatalytic device comprising base layer 119, proximal electrode layer 121, electroluminescent layer 123, distal electrode layer 125, and photocatalytic layer 127. Base layer 119 may be the surface of a medical implant or an insulating layer. Proximal electrode layer 121, electroluminescent layer 123, distal electrode layer 125, and photocatalytic layer 127 may be deposited by chemical vapor deposition techniques such as atomic layer disposition (ALD), atomic layer epitaxy (ALE), assisted CVD, and metalorganic vapor phase epitaxy; physical vapor deposition techniques such as high velocity oxygen fuel, pulsed laser deposition, sputtering, arc-PVD, EBPVD, plasma spraying, electroplating, and low-pressure plasma spraying (LPPS); other techniques such as evaporation, ion beam assisted deposition (IBAD), magnetron sputtering, molecular beam epitaxy, slurry or dye techniques, sintering technique, sol-gel, and sputter ion plating; and other techniques known to those of skill in the art or combinations thereof.

Upon excitation via an alternating electric charge, electroluminescent layer 123 illuminates photocatalytic layer 127 from below to promote photocatalysis. The use of electroluminescent layer 123 as a light source is advantageous because it may be deposited on to complex three-dimensional surfaces in a variety of ways, such as spraying, and may also be more efficient and effective than other means known in the art for illuminating complex three-dimensional surfaces. The electroluminescent layer may comprise any fluorescent or electroluminescent materials known to those of skill in the art and may further comprise phosphors or quantum dots.

Proximal electrode layer 121 may comprise transparent conductive oxides such as indium or tin oxides (such as SnO₂ or In₂O₃) or doped combinations thereof, carbon nanotube films, conductive polymers, metal dispersions, conductive composite materials, colloidal silver or mixtures thereof Proximal electrode layer 121 may further comprise thin layers of conductive media or fine conductive meshes that do not obscure the net flux of outward illumination nor hinder the detection of an incoming signal. It will be appreciated by those of skill in the art that proximal electrode layer 121 may be chosen to ensure high transparency to the desired wavelengths of electromagnetic radiation and may have high electrical conductivity. Furthermore, proximal electrode layer 121 may comprise materials such as reflective metal or carbon if non-transparency is desired.

Distal electrode layer 125 may comprise an optically transparent electrically conducting oxide layer that may act as a cap layer for the electroluminescent layer 123 and as an electrode for the purpose of electrically biasing the photocatalytic layer 127 to retard electron-hole recombination. The distal electrode layer 125 may comprise the same materials as disclosed above with reference to proximal electrode 121, with the exception of non-transparent materials. The distal electrode layer 125 promotes charge separation by attracting electrons toward its positively charged upper surface, thereby biasing the photocatalytic layer 127 and retarding electron-hole recombination. For the purpose of electrically biasing the photocatalytic layer 127, the in vivo environment may be used as a ground that may be equivalent to a negative terminal. Also, the distal electrode layer 125 may comprise two optically transparent electrically conducting layers separated by an additional optically transparent electrically insulating layer, whereby the bias may be locally bipolar and the use of in vivo grounding may be avoided (not shown).

Electrically biasing the photocatalytic layer increases the energy efficiency of the photocatalytic reactions and increases the amount of organic material destroyed or prevented from attaching to the photocatalytic layer. Photocatalytic activity is difficult to measure directly; consequently, it is typically inferred indirectly by equivalence to the absolute or relative rate of a photocatalytic reaction, often via observing the extent and rate of degradation of organic dyes. Coating a working electrode with thin films of titania and tin oxide, followed by UV irradiation, increases the efficiency of the selective oxidation of organic compounds such as azo dyes. Indeed, results from K. Vinodgopal and P. V. Kamat indicate an 8-fold increase in efficiency of the oxidation of an azo dye using a TiO₂/SnO₂ nanocomposite versus a TiO₂ control. K. Vinodgopal and Prashant V. Karnat, K. Vinodgopal and P. V. Kamat, Environ. Sci. Technol. 29 (1995) 841. Moreover, results from Taicheng An et al., indicated a 21.8% increase in decolorization of methyl blue versus a TiO₂ control. Taicheng An, Guiying Li, Ya Xiong, Xihai Zhu, Hengtai Xing and Guoguang Liu, Mater. Phys. Mech. 4 (2001) 101-106.

The energy efficiency of photocatalytic reactions may also be improved through the use of composites including nano-scale composites employing catalytic agents in combination with a metals. Modification of a semiconductor with a noble metal may be beneficial for promoting charge transfer from a photo-excited semiconductor. Charge transfer to the metal from the semiconductor modifies the energetics of the composite by shifting the Fermi level to a more negative potential, thereby promoting charge separation and improving the catalytic activity of the composite catalyst.

The catalytic agents may comprise semiconductors or Perovskite compounds such as SrTiO₃, or other compounds known to exhibit photocatalytic behavior. The metals may comprise platinum group metals, silver, gold, aluminum, iron, or mixtures thereof. The composites may be in the form of coated particles or shelled particles (e.g. a metal core with a semiconductor shell or a semiconductor core with a metal shell), laminates, or dispersed composite mixtures. Semiconductor-metal composites may comprise for example, TiO₂—Au, ZnO—Pt, or TiO₂—CdSe. Perovskite-metal composites may comprise for example, compounds of the formula Sr₍₁₋ₓ₎Ag₍ₓ₎TiO₃.

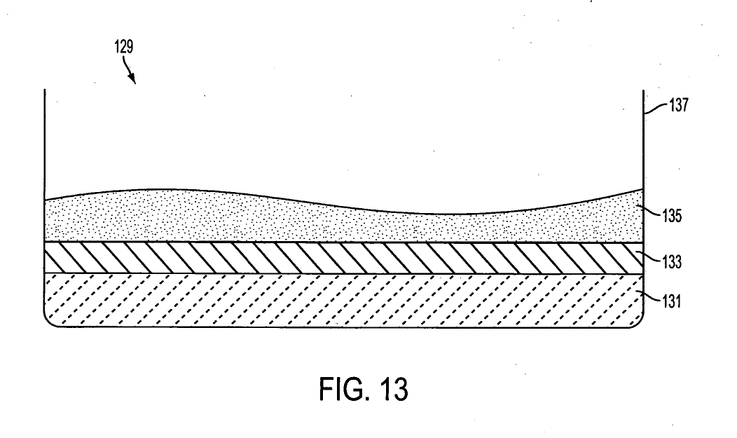

Referring to FIG. 13, there is shown a tissue scaffold 129 comprising a base layer 131 and sides 137. A photocatalytic layer 133 comprising a semiconductor oxide such as TiO₂ may be supported by base layer 131. Tissue layer 135 represents living cellular tissue growing on the surface of photocatalytic layer 133. Upon illumination of photocatalytic layer 133 by electromagnetic radiation such as UV or visible light, this layer becomes hydroxylated and superhydrophilic, which aids in the release of tissue layer 135 from tissue scaffold 129.

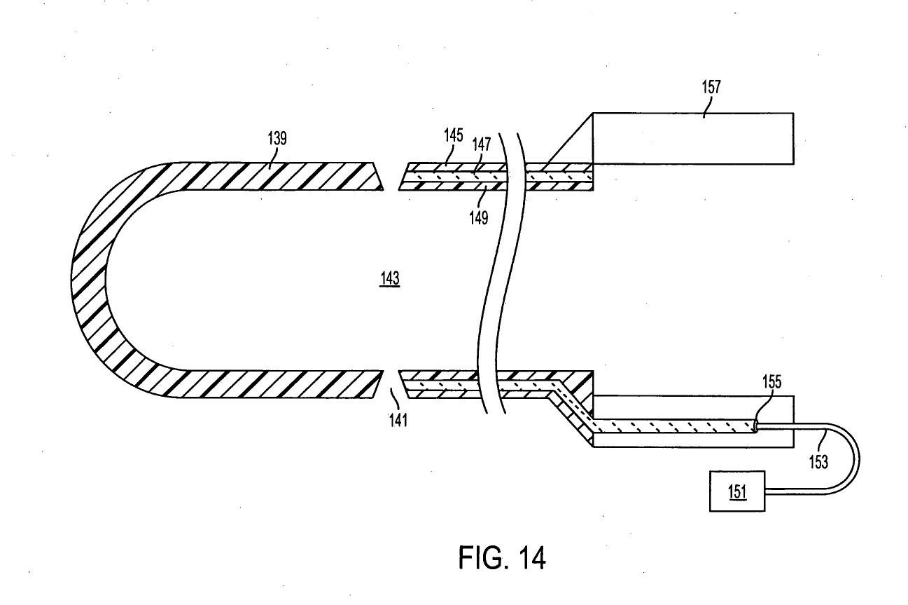

Referring now to FIG. 14, there is shown a catheter having a catheter tip 139, catheter wall 149, opening 141, lumen 143, and catheter adaptor 157. The sides of the catheter comprise catheter wall 149 supporting waveguide layer 147 and photocatalytic layer 145. Light from light source 151 travels through fiber optic cable 153 to light port 155, where it enters waveguide 147 to be dispersed to photocatalytic layer 145. Catheter tip 139 and catheter wall 149 may be comprised of conventional polymer or rubber materials known to those of skill in the art. Photocatalytic layer 145 comprises a semiconductor oxide such as TiO₂ that upon illumination with UV or visible light becomes hydroxylated and superhydrophilic.

It will be appreciated that fiber optic cable 153 may comprise a circular array of fiber optics or a circular configuration fiber optics such as a tubular optical cable, wherein the fiber is hollow (not shown) and may be adapted to evenly distribute light to waveguide layer 147. It will further be appreciated that light source 151 may be incorporated into the catheter.

The photocatalytic layer 145 may be activated (i.e. made superhydrophilic or “slippery” through the use of electromagnetic radiation) to ease insertion of the catheter. Once the catheter is in the desired position, the light source 151 may be switched off so that the photocatalytic layer 145 loses its photo-induced superhydrophilicity and the catheter may be held in place by friction. Upon desired removal of the catheter, the light source 151 may be turned on to ease removal of the catheter.

It will be appreciated by those of skill in the art that the various embodiments of this invention are not limited to drainage catheters and may also be employed in therapy delivery catheters, hydrocephalus shunts, ablation catheters, pacing leads, or other tubular medical devices. It is further contemplated that multiple photocatalytic layers could be disposed lengthwise about the circumference of the catheter and individually activated to create a more or less superhydrophilic surface as necessary to steer a catheter to the desired location in the body. It is further contemplated that more than one light source could be used in some embodiments.

In some embodiments, illumination of a photocatalytic layer such as TiO₂ with ultraviolet or visible light may be employed for delivering therapeutic agents. In some embodiments, the reactive oxygen species produced by photocatalysis act to cleave bonds and release therapeutic agents attached to the photocatalytic surface. In some embodiments, therapeutic agents may be released by controlled changes in the superhydrophilicity or hydrophobicity of the photocatalytic layer. In this way, controlled elution of therapeutic agents from the photocatalytic surface may be produced in vivo by controlling the amount of electromagnetic radiation applied to the photocatalytic layer. Therapeutic agents capable of being delivered in this manner include drugs, proteins, DNA, siRNA, and viruses that are modified to deliver a therapeutic gene. Indeed, any of the following therapeutic agents, alone or in combination may be delivered according to some embodiments of the invention: anti-proliferative agents, anti-inflammatory agents, cell suspensions, polypeptides which is used herein to encompass a polymer of L- or D-amino acids of any length including peptides, oligopeptides, proteins, enzymes, hormones and the like, immune-suppressants, monoclonal antibodies, polynucleotides which is used herein to encompass a polymer of nucleic acids of any length including oligonucleotides, single- and double-stranded DNA, single- and double-stranded RNA, iRNA, DNA/RNA chimeras and the like, saccharides, e.g., mono-, di-, poly-saccharides, and mucopolysaccharides, vitamins, viral agents, and other living material, radionuclides, and the like, antithrombogenic and anticoagulant agents, antimicrobial agents such as antibiotics, antiplatelet agents and antimitotics, i.e., cytotoxic agents, and antimetabolites.

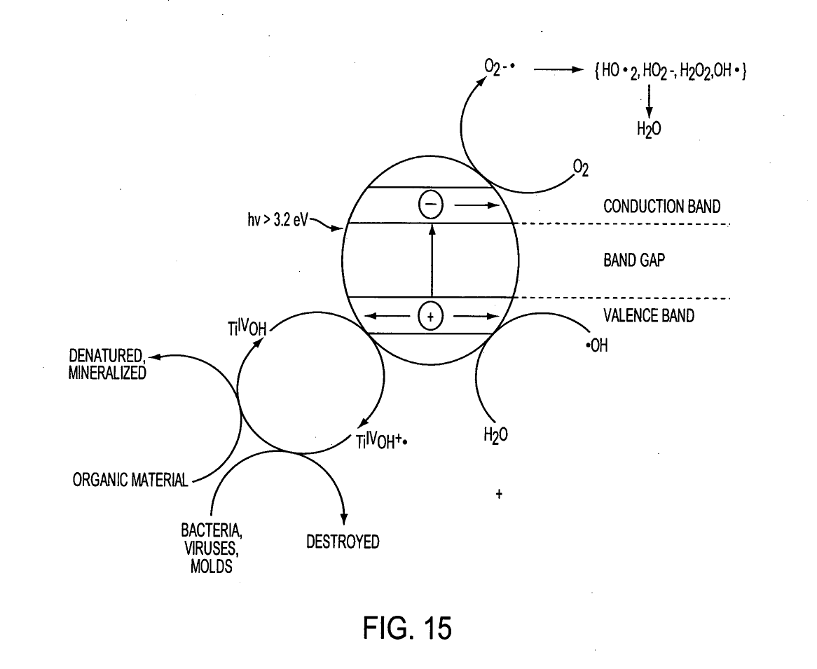

An experiment demonstrates that photocatalysis may be used to eliminate organic material. Specifically, FIG. 15 provides a schematic illustrating the reaction mechanisms leading to pronounced photocatalysis and superhydrophilicity. As this schematic demonstrates, titanium dioxide (TiO₂) in appropriate forms (e.g., thin-films of anatase) may exhibit pronounced photocatalytic and superhydrophilic behavior when irradiated with specific wavelengths of electromagnetic radiation. Photocatalysis then has the effect of preventing, reducing and removing organic matter attached at the surface of a medical device, such as a window on a medical device that would otherwise be obstructed. Keeping medical device surfaces clear thus leads to prolonged implant functional life and performance.

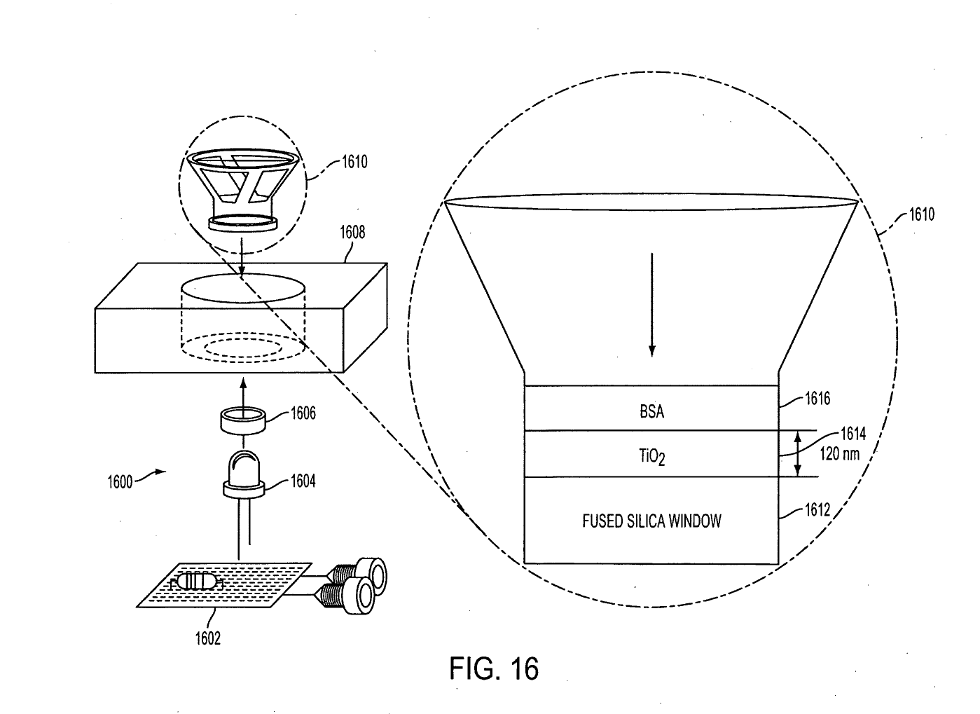

FIG. 16 depicts an experimental device 1600 that provides a circuit board 1602 on which a light source (in this case an LED) 1604 has been provided. A ring 1606 is provided to secure in place a cell well insert 1610 that has been disposed within a container 1608. The cell well insert 1610 adjoins a fused silica window 1612 with a layer of TiO₂ 1614 deposited onto fused silica window 1612 up to the base of cell well insert 1610. Cell well insert(s) 1610 were then placed directly above LEDs 1604, which irradiated the TiO₂ surface at a wavelength of 365 nm (UV). Aliquots of fluorescently labeled bovine serum albumin (BSA) in solution 1616 were added to the cell well inserts, covering the TiO₂ coated surface.

Results of this experiment revealed that BSA adhered to control surfaces of (both TiO₂ coated non-illuminated, and non-coated UV illuminated) after a post rinse with phosphate buffered saline (PBS), whereas the UV illuminated TiO₂ specimens exhibited a central region significantly depleted in BSA—coincident with the region of UV illumination. This experiment may be repeated with comparable results.

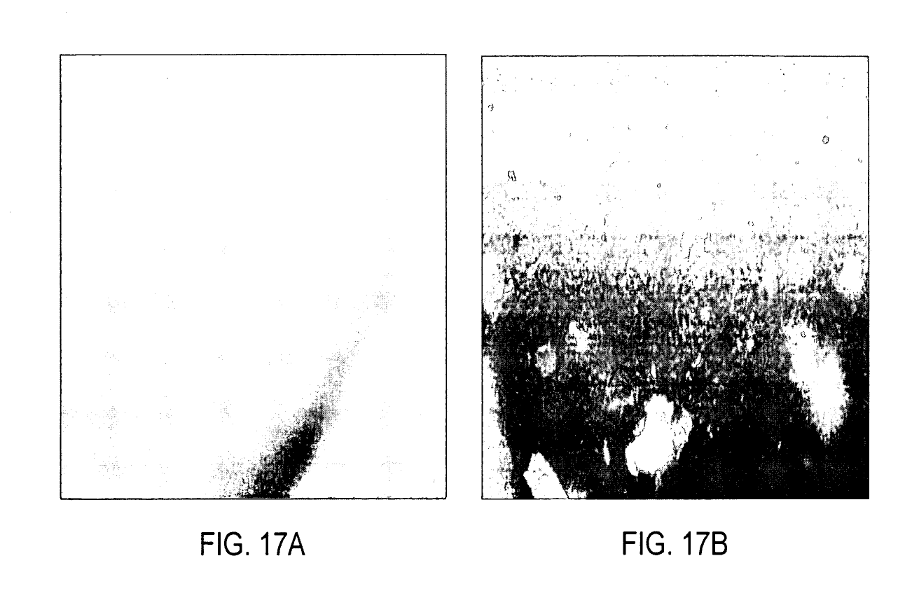

FIGS. 17(a) and 17(b) demonstrate a comparison in photograph of a control surface (in FIG. 17(a)) with illuminated surface (in FIG. 17(b)). As these photographs demonstrate, illuminated surfaces are significantly depleted of BSA near the center where illumination took place.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Further, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

1. An implant comprising:
a photocatalytic layer disposed on an electrically conductive layer, wherein the electrically conductive layer is adapted to be electrically biased.

2. The implant of claim 1, further comprising an insulating layer located between the photocatalytic layer and the electrically conductive layer.

3. The implant of claim 1, further comprising an insulating layer, wherein the electrically conductive layer is located between the photocatalytic layer and the insulating layer.

4. The implant of claim 1, further comprising a voltage source adapted to electrically bias the electrically conductive layer.

5. The implant of claim 1, wherein the photocatalytic layer comprises a laminate, and wherein the laminate comprises at least one metal and at least one catalytic agent.

6. The implant of claim 1, wherein the photocatalytic layer comprises a composite, and wherein the composite comprises at least one metal and at least one catalytic agent.

7. The implant of claim 6, wherein the composite comprises shelled particles or coated particles or mixtures thereof.

8-10. (canceled)

11. The implant of claim 1, wherein the electrically conductive layer comprises SnO₂, In₂O₃, carbon nanotubes, conductive polymers, metal dispersions, conductive composite materials, colloidal silver, or mixtures thereof.

12. The implant of claim 1, further comprising a light sensitive diode adapted to receive a signal from outside the implant.

13. The implant of claim 1, further comprising an induction coil connected to a rechargeable battery.

14. The implant of claim 1, further comprising a circuit board comprising a telemetry coil.

15. The implant of claim 14, wherein the circuit board is adapted to communicate with an external device.

16. The implant of claim 1, wherein the implant is at least partially enclosed by a housing comprising a hermetic seal, wherein the housing is electrically grounded by an in vivo environment contacting the housing.

17. The implant of claim 1, wherein the photocatalytic layer comprises a material selected from the group consisting of TiO₂, NaTaO₃, ZnO, CdS, GaP, SiC, WO₃, ZnS, CdSe, SrTiO₃, CaTiO₃, KTaO₃, Ta₂O₅, ZrO₂, doped or non-doped, sensitized or non-sensitized, and mixtures thereof.

18. The implant of claim 1, wherein the implant comprises a sensor selected from the group consisting of an oxygen sensor, an electromagnetic radiation sensor, an impedance sensor, a pressure sensor, a protein sensor and a glucose sensor.

19. The implant of claim 18, wherein the sensor is an oxygen sensor

20. The implant of claim 1, wherein the implant comprises a spectroscopy device.

21. The implant of claim 1, further comprising a sensor window.

22-27. (canceled)

28. A method comprising:
providing a medical implant comprising a photocatalytic layer and an electrically conductive layer;
illuminating the photocatalytic layer with a light source; and
electrically biasing the electrically conductive layer.

29-36. (canceled)

37. An implant comprising:
a photocatalytic layer; and
an electroluminescent layer.

38-43. (canceled)

44. A method comprising:
disposing an electroluminescent layer on a medical implant; and
illuminating a photocatalytic layer disposed on the medical implant with light from the electroluminescent layer.

45. The method of claim 44, wherein an electrode layer is disposed between the photocatalytic layer and the electroluminescent layer.

46. A method for controlled delivery of a therapeutic agent comprising:
providing a medical implant having one or more therapeutic agents disposed on a photocatalytic layer on the implant, and
illuminating the photocatalytic layer with electromagnetic radiation;
wherein the therapeutic agent comprises a drug, a protein, DNA, siRNA, or a virus that is modified to deliver a therapeutic gene, or mixtures thereof.

47. The method of claim 46, wherein the therapeutic agent is disposed within a matrix or discrete reservoir.

48. The method of claim 46, wherein the therapeutic agent is released by the photocatalytic reaction.
